(19) United States
(12) Patent Application Publication
(10) Pub. No.: US 2010/0114126 A1
(43) Pub. Date: May 6, 2010

(54) MAGNETIC POSITIONING OF SURGICAL MESH

(76) Inventor: Marc Neff, Cherry Hill, NJ (US)

Correspondence Address: ITALIA IP, 3500 WEST OLIVE AVE., SUITE 300, BURBANK, CA, 91505, US

(21) Appl. No.: 12/263,491

(22) Filed: Nov. 2, 2008

(51) Int. Cl. A61B 17/08

(52) U.S. Cl. 606/151

(57) ABSTRACT

A method of appropriately positioning a surgical mesh relative to body tissue during surgery, using at least two magnets or alternatively, at least a magnet and a magnetically responsive material. The magnet or magnetically responsive materials are delivered to the surgical site for example through a laparoscope. A magnet or magnetically responsive material may be in sheet form and coiled or otherwise made compact to accommodate the laparoscope, then expanded to a final effective configuration. If the surgical mesh is not appropriately located relative to the body tissue, it may be appropriately repositioned by urging it using a tool, which may act by magnetic attraction, such that the mesh slides along the body tissue. The surgical mesh may then be fixed to body tissue using conventional surgical fasteners.

### FIELD OF THE INVENTION

The present invention relates to manipulating a surgical mesh into appropriate positioning on a patient during surgery.

### BACKGROUND OF THE INVENTION

Surgical mesh material must be accurately placed against tissues of a patient in the course of surgery, such as for example against the abdominal wall for laparoscopic ventral or incisional hernia repair. Ordinarily, in a minimally invasive procedure wherein only small instruments are used, meshes are manipulated with visual feedback provided by a video camera. Once positioned, the mesh may be secured in place by tacks, screws, and sutures. However, the environment of surgery wherein minimally invasive techniques are employed, is fraught with problems in manipulating surgical mesh materials. Notably, working space in the surgical environment is quite limited. The mesh may be relatively large and therefore more difficult to orient appropriately. The view from the video camera may be less than ideal due to angle of the viewing camera. As a consequence, the task of positioning a mesh may become prolonged or may entail other problems such as increasing chances of recurrence because of poor centering for example, on a hernia.

### SUMMARY OF THE INVENTION

The present invention reduces the effort and difficulty of placing a surgical mesh. The mesh may be pinned magnetically in place against the abdominal wall by sandwiching the mesh and tissue between two magnetically responsive members. One of these members must be a magnet and the other may be either magnetic or merely magnetically responsive. The magnetic members may be relatively small, and therefore may be easily maneuvered into a suitable position. The magnetically responsive members are neither part of nor fixed to the mesh itself. Therefore, the magnetically responsive materials may be easily withdrawn from the surgical site when the mesh is suitably fixed in place.

The novel concept may be better understood with reference to the following analogy. A sheet of ordinary paper may be pinned against a glass sheet using magnets placed outside both the glass and the paper in sandwich fashion. The paper may then be readily repositioned by moving the magnets appropriately, so that the paper and magnets glide along the surface of the glass.

It is an object of the invention to use magnetically responsive members to position a mesh suitably at a surgical site.

Another object of the invention is to use objects to hold a mesh in place which are easily maneuvered in placing the surgical mesh, and readily withdrawn when the mesh is in place.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is an environmental end view of a surgical mesh entrapped against body tissue by magnetic members.

FIG. 2 is a plan view of a magnetic member configured according to one aspect of the invention.

FIG. 3 is a plan view of a magnetic member configured according to another aspect of the invention.

FIG. 4 is a perspective view of a magnetic member configured according to another aspect of the invention.

FIG. 5 is a block diagram of steps of a method of positioning a surgical mesh magnetically, according to at least one aspect of the invention.

### DETAILED DESCRIPTION

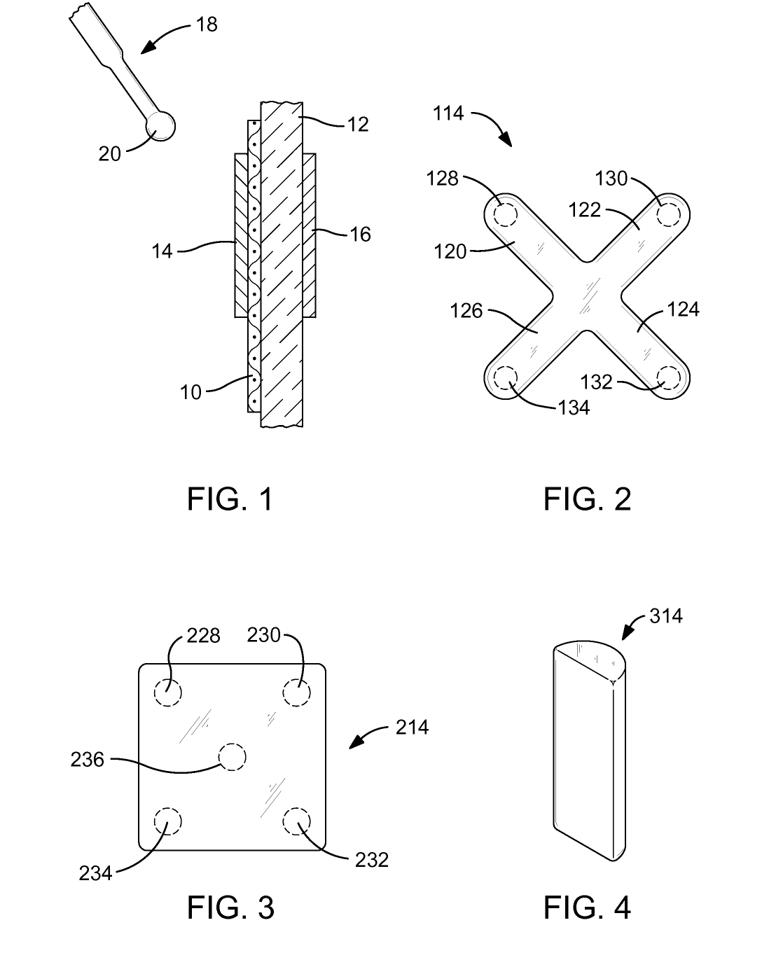

FIG. 1 of the drawings shows a surgical mesh 10 pinned against a body tissue 12, such as an abdominal wall, by a first magnetic material 14 and a second magnetic material 16. The first magnetic material 14 and the second magnetic material 16, one of which must be a magnet and the other of which may be either a magnet or merely magnetically responsive, have been inserted into place using conventional surgical tools such as a laparoscope (not shown). The surgical mesh 10 may be maneuvered into a final position relative to the body tissue 12 by other conventional surgical tools (not shown).

The advantage of using magnetic materials such as the first magnetic material 14 and the second magnetic material 16 is that repositioning of the surgical mesh is relatively easily accomplished. The first magnetic material 14 and the second magnetic material 16 are small enough to be readily delivered to the surgical site and removed therefrom by for example by a laparoscopic tool.

FIG. 2 shows a magnetic material member 114, which may comprise a flexible sheet formed to have four arms 120, 122, 124, 126. The magnetic material member 114 may be for example, a plastic or elastomeric material impregnated with magnetically responsive material, such as comminuted metal. Magnets 128, 130, 132, 134 may be contained within the arms 120, 122, 124, 126 at the outer ends thereof. The advantage of configuring the magnetic material member 114 as a cruciform as shown in FIG. 2 is that a cruciform can be rolled up or coiled, or otherwise put into a compact configuration, and in that configuration, can be delivered relatively easily using a laparoscopic tool. The magnetic material member 114 may be withdrawn from the surgical site after surgery using the same laparoscopic tool, after being twisted, recoiled, or otherwise reconfigured into a compact condition to accommodate the laparoscopic tool.

The surgical mesh material 10 is shown supported against the body tissue 12. It may be that the surgical mesh material 10 has been appropriately placed against the body tissue 12 during initial installation, in which case the surgical mesh material 10 may be secured to the body tissue using conventional securement methods such as tacking, suturing, and screwing. If placement of the surgical mesh material 10 is not as desired by the surgeon, then the surgical mesh material 10 may be maneuvered into a final or desired position or location using a tool to maneuver at least one of the first magnetically responsive material 14 and the second magnetically responsive material 16. As depicted in FIG. 1, a tool such as the tool 18 may have a magnetic member 20 fixed thereto, to enable moving the first magnetically responsive member 14 using magnetic attraction. Of course, the first magnetic material 14 and the second magnetic material 16 may if desired by moved by interference or other mechanical engagement, rather than using magnetic attraction.

The magnetic member 114 exemplifies one of many different configurations which may be assumed. FIG. 3 shows another configuration, in which a magnetic member 214 is rectangular. The magnetic member 214 may have internally contained magnets 228, 230, 232, 234, and 236. The magnetic member 214 may be of area or footprint that is less than that of the associated surgical mesh, such as the surgical mesh 10. This relationship assures that the surgical mesh will be exposed outside the bounds of the magnetic member 214, thereby presenting surface area for receiving fasteners to permit attachment to body tissue with the supporting magnetic material remaining in place. Other than leaving a suitable exposed border for receiving medical fasteners, it is preferred that the magnetic member approach the area or footprint of the surgical mesh to assist in supporting and maneuvering the surgical mesh, as will be further discussed hereinafter.

It should be noted that while the magnetic members 114 and 214 are shown as comprising magnets 128, 130, 132, 134, 228, 230, 232, 234, and 236 located at discrete positions, it would be possible to utilize an elastomeric material or for example a silicone sheet which is impregnated throughout with magnetic or magnetically responsive particles, rather than using discrete individual magnets.

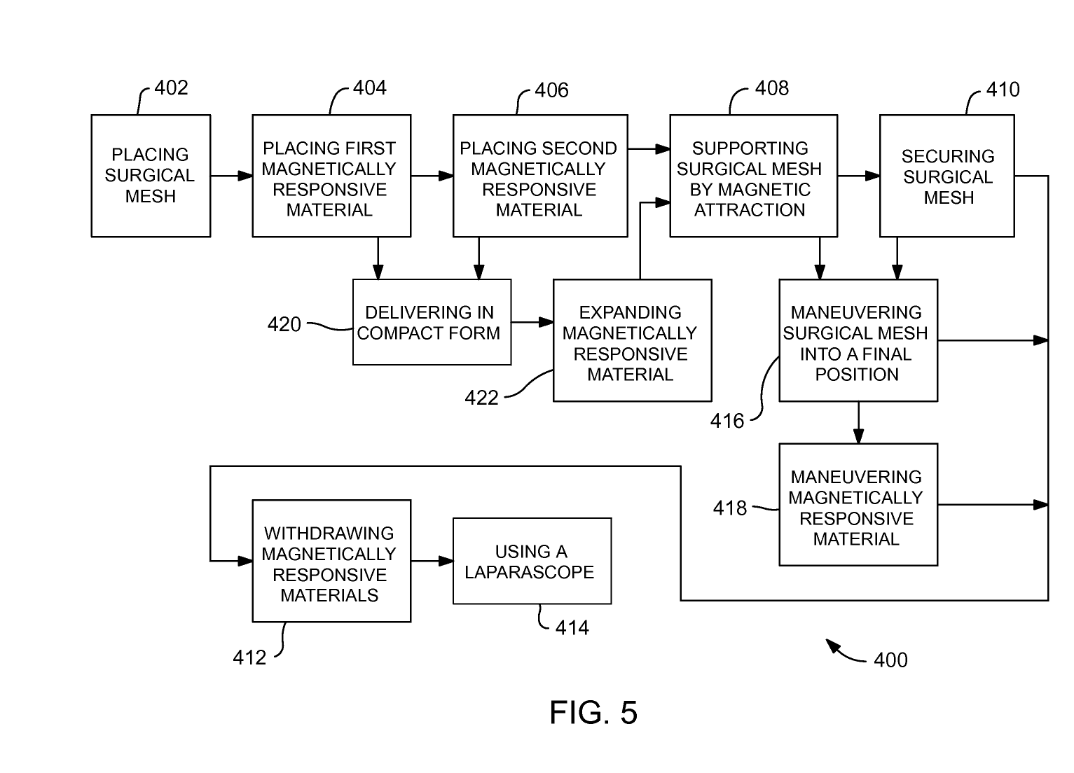

FIG. 4 illustrates a straight magnetic member 314 which may be employed. The magnetic member 314 may be dimensioned and configured to slide through the tubing of a laparoscopic tool such as that used to deliver magnetically responsive materials to the surgical site, and therefore may be rigid or substantially firm holding rather than flaccid or flexible, as are the magnetic members 114 and 214. The magnetic member 314 may be for example fifteen millimeters in length.

FIG. 5 shows a method 400 of appropriately placing and securing surgical mesh, such as the mesh 10 or 110 in place against body tissue, such as the body tissue 12, during a surgical procedure. The method 400 may comprise a step 402 of placing a surgical mesh material, such as the surgical mesh material 10, against the body tissue, such as the body tissue 12, as seen in FIG. 1.

The method 400 may comprise a step 404 of placing a first magnetically responsive member, such as the magnetically responsive member 14 into the surgical site on one side of the surgical mesh material and the body tissue, and a step 406 of placing a second magnetically responsive member, such as the magnetically responsive member 16, into the surgical site on the other side of the surgical mesh material and the body tissue. The surgical mesh material are thereby sandwiched the surgical mesh material and the body tissue. As has been stated prior, at least one of the first magnetically responsive material and the second magnetically responsive material is a magnet.

The method 400 may comprise a step 408 of initially supporting the surgical mesh material relative to the body tissue by magnetic attraction, such as by using the first magnetically responsive material 14 and the second magnetically responsive material 16.

The method 400 may comprise a step 410 of securing the surgical mesh material to the body tissue, as may be done for example using conventional surgical fasteners.

The method 400 may comprise a step 412 of withdrawing from the surgical site the first magnetically responsive member and the second magnetically responsive member. This may be done for example using the same laparoscopic tool used to deliver the first magnetically responsive member and the second magnetically responsive member to the surgical site. This is seen as a step 414.

While it is possible that the surgical mesh may have been appropriately placed immediately upon delivery to the surgical site, it is far more likely that some adjustment to positioning of the surgical mesh be required. Therefore, the method 400 may comprise a step 416 of maneuvering the surgical mesh material into a final desired position relative to the body tissue while the surgical mesh material is at least partially supported by the first magnetically responsive material and the second magnetically responsive material.

In maneuvering the surgical mesh, should positional adjustment be necessary, it is possible to maneuver at least one of the first magnetically responsive material and the second magnetically responsive material along the body tissue such that magnetic attraction moves the surgical mesh material. This is seen as a step 418 of the method 400.

Where at least one of the first magnetically responsive material and the second magnetically responsive material is provided as a flexible sheet, the method 400 may comprise a further step 420 of delivering the flexible sheet to the surgical site in a compact form, and a further step 422 of expanding the flexible sheet to a spread condition once the flexible sheet is at the surgical site. It will be appreciated that magnetically responsive materials which are deformable to expand from a compact configuration to a spread configuration are susceptible to being twisted or otherwise manipulated to assume a compact configuration to accommodate withdrawal as the surgical procedure nears completion.

The method 400 may employ magnetically responsive materials provided both as relatively flat, thin, flexible sheets, and also as linear members, as seen in FIG. 4.

It should be stressed that the steps of the method 400 need not necessarily be performed in the order shown above. For example, it may be possible to deliver magnetically responsive materials to the surgical site before delivering the surgical mesh thereto.

Also, the nature, number, and configuration of the magnetically responsive materials may be varied to suit. Illustratively, a number of individual, separate magnets may be employed in place of a single, relatively large member such as the magnetic member 114 or the magnetic member 214.

Magnetic members may be uniformly provided with magnetic characteristics, only at the periphery, periodically along the periphery, and at the center, in continuous or discontinuous fashion.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A method of securing surgical mesh in place against body tissue during a surgical procedure, comprising the steps of:
placing a surgical mesh material against the body tissue;
placing a first magnetically responsive member into the surgical site on one side of the surgical mesh material and the body tissue;
placing a second magnetically responsive member into the surgical site on the other side of the surgical mesh material and the body tissue, thereby sandwiching the surgical mesh material and the body tissue, wherein at least one of the first magnetically responsive material and the second magnetically responsive material is a magnet;
supporting the surgical mesh material relative to the body tissue by magnetic attraction;
securing the surgical mesh material to the body tissue; and
withdrawing from the surgical site the first magnetically responsive member and the second magnetically responsive member.

2. The method of claim 1, comprising the further step of maneuvering the surgical mesh material into a final desired position relative to the body tissue while the surgical mesh material is at least partially supported by the first magnetically responsive material and the second magnetically responsive material.

3. The method of claim 2, wherein the step of maneuvering the surgical mesh material into a final desired position comprises the further step of maneuvering at least one of the first magnetically responsive material and the second magnetically responsive material along the body tissue such that magnetic attraction moves the surgical mesh material.

4. The method of claim 1, wherein at least one of the first magnetically responsive material and the second magnetically responsive material is a flexible sheet, comprising the further steps of delivering the flexible sheet to the surgical site in a compact form, and expanding the flexible sheet to a spread condition once the flexible sheet is at the surgical site.

5. The method of claim 1, comprising the further step of using a laparascopic tool to deliver the first magnetically responsive material and the second magnetically responsive material to the surgical site.

6. The method of claim 1, wherein the step of securing the surgical mesh material to the body tissue comprises the further step of fixing the medical mesh in place at a desired orientation by using a medical fastener.

7. The method of claim 1, wherein one of the first magnetically responsive material and the second magnetically responsive material is a flexible sheet, and further comprising the step of delivering the flexible sheet to the surgical site in a compact form, and expanding the flexible sheet to a spread condition once the flexible sheet is at the surgical site.

8. The method of claim 1, wherein one of the first magnetically responsive material and the second magnetically responsive material is a linear member.

9. The method of claim 1, wherein the magnet is no greater than about fifteen millimeters in length.
